(19) United States
(12) Patent Application Publication
(10) Pub. No.: US 2026/0308500 A1
(43) Pub. Date: Oct. 8, 2026

(54) IMPROVEMENTS IN CELL CULTURE

(71) Applicant: CN BIO INNOVATIONS LIMITED, Cambridge (GB)

(72) Inventors: Yassen ABBAS, Cambridge (GB); Daniel CARNEY, Cambridge (GB); Alberto GARCIA NARANJO, Cambridge (GB); Uranbileg MYAGMARSUREN, Cambridge (GB)

(73) Assignee: CN BIO INNOVATIONS LIMITED, Cambridge (GB)

(21) Appl. No.: 19/483,185

(22) PCT Filed: May 15, 2024
(86) PCT No.: PCT/GB2024/051268

§ 371 Date: Nov. 10, 2025

(30) Foreign Application Priority Data
May 18, 2023 (GB) 2307455.2

(51) Int. Cl. C12M 1/32; C12M 1/00; C12M 3/00

(52) U.S. Cl. CPC C12M 23/12; C12M 23/38; C12M 23/44; C12M 23/48; C12M 23/50

(57) ABSTRACT

The present invention relates a device for culturing cells, particularly a cell culture component for use with a cell culture system. The invention also relates to a cell culture system.

### FIELD OF THE INVENTION

The present invention relates to improvements in cell culture, and in particular to an improved device for culturing cells in a cell culture insert.

### BACKGROUND TO THE INVENTION

Cell culture inserts are used in conjunction with well plates for the growth and differentiation of various cell types. Cell culture inserts are suspended within a well, and provide a permeable or semi-permeable membrane dividing the well into two compartments. Cells may be cultured on the membrane which divides the well into apical and basal domains; this makes it possible to study response of cells to different conditions in the apical and basal domain, or to determine the response of a cellular monolayer on both sides of the membrane.

In order to provide preferred conditions on the apical domain of the well, various solutions have been developed which can provide culture medium or other growth components to the apical domain. Note that typically the basal domain is relatively straightforward to access, being formed of the well plate itself in combination with the cell culture insert. One such solution is an apical plug which allows culture medium to flow over the apical surface of the cell culture insert. An example is that described in U.S. Pat. No. 10,323,221, which describes an apical plug to be inserted within the cell culture insert, sealing against its interior, such that a discrete environment on the apical side can be created and cell culture media flowed through, perfusing the cell culture on the apical side.

Critical to successful culturing of cells is the maintaining of a sterile environment during the experiment. With cell culture inserts this is typically achieved by handling within a sterile microbiological safety cabinet for any manipulation and, at all other times, covering the plate in which the cell culture wells and inserts are located with a solid lid. However, with an apical plug in place, unless the plug is of restricted height, these standard lids cannot be used.

In the case of the apical plug described in U.S. Pat. No. 10,323,221, the culture well is covered by the plug itself, which has a diameter greater than that of the cell culture insert. However, with this design it is not possible to access media on the basal side of the cell culture insert, in the cell culture well itself, without completely removing the plug and cell culture insert assembly to expose the culture well. Additionally, removing the apical plug from the cell culture insert at an experiment's conclusion is likely to require handling of the cell culture insert body, near to the cell-supporting membrane, increasing risk of contamination and damage to the cell culture.

There is therefore a need for alternative structures, which will permit the use of apical plugs without the drawbacks of prior art devices.

### SUMMARY OF THE INVENTION

According to a first aspect of the present invention, there is provided a cell culture component for use with a cell culture system having a cell culture well, a cell culture insert received in the cell culture well, and an apical plug received in the cell culture insert, the cell culture component comprising:
a bracket defining an aperture within which an apical plug may be received, the bracket having an outer diameter larger than that of the cell culture insert, and the aperture having a diameter smaller than that of the cell culture insert; wherein the aperture is sized and shaped so as to define a first region which will be closed by an apical plug when the plug is received within the aperture, and a second region which will remain open when the apical plug is received within the aperture; and
a lid to be received on the bracket and defining an aperture corresponding with the bracket aperture, within which the apical plug may be received;
wherein the lid is rotatable relative to the bracket so as to align or unalign the second regions of the respective apertures, so as to open or close a path through the lid and bracket.

The combination of the bracket and lid allow the use of a narrower apical plug in a cell culture insert, which plug does not necessarily have to seal the whole of the culture well. The path through the lid and bracket created by aligning the apertures of the two components will bypass the plug and allow ready access to the cell culture media located on the apical side of the cell culture insert barrier, in the cell culture well, while keeping the environment sterile by not leaving the well exposed and open to potential contaminants.

Further, the configuration of the bracket as being larger in outer diameter than the cell culture insert means that the bracket may be held in place to allow easy removal of an apical plug without disturbing the cell culture insert, which insert will be retained by the bracket. In addition, if retained manually by a user, then having a larger-sized bracket ensures that the user's fingers are sufficiently distant from the cell culture insert membrane and cells cultured on it to reduce the risk of disturbance of the cells or contamination.

The bracket aperture is preferably asymmetric, such that the second region is offset from the first region, and does not extend around the whole of the first region. In a preferred embodiment, the bracket aperture is bilobate; for example, the aperture may include a first, larger region for receiving the apical plug, and a second, offset, smaller region for forming the path. The first and second regions of the aperture are preferably contiguous, although this need not be the case in all embodiments.

The lid aperture which corresponds with the bracket aperture need not be identical to the bracket aperture; all that is necessary is that the lid aperture includes regions which will align with the corresponding bracket aperture regions sufficiently to provide continuous openings for the apical plug and path respectively. The corresponding regions may be of different sizes and/or shapes. In one embodiment, the lid may be generally annular; in this embodiment, the first lid aperture region may be a central annular opening, and the second aperture region may be in the form of a notch from the rim of the annulus. The notch may extend completely through the rim, such that the lid is discontinuous.

Preferably the first regions of the lid aperture and the bracket aperture are concentric; this allows the lid to rotate on the bracket to align the second regions in a straightforward manner.

In preferred embodiments, the bracket is able to be fixed to the culture well. In embodiments, the bracket may comprise one or more fittings for allowing it to be secured to a culture well. These may take the form of one or more holes for receiving screws; the bracket may also comprise the relevant screws. Other suitable fittings may be used; for example, pins, hooks, interference fittings, and so on.

In some embodiments, the bracket may comprise a plurality of apertures as defined; preferably two such apertures. Here the bracket may take a bilobate or FIG. 8 shape.

This allows a single bracket to cover multiple (for example, two) culture wells. A corresponding plurality of lids may be provided.

In some embodiments, one or more of a cell culture well, a cell culture insert, and an apical plug may also be provided.

A further aspect of the invention provides a cell culture system comprising a cell culture well, a cell culture insert received in the cell culture well, and an apical plug received in the cell culture insert, the cell system further comprising:
a bracket to be located on the cell culture well and defining an aperture within which the apical plug may be received, the bracket having an outer diameter larger than that of the cell culture insert, and the aperture having a diameter smaller than that of the cell culture insert; wherein the aperture is sized and shaped so as to define a first region which will be closed by the apical plug when the plug is received within the aperture, and a second region which will remain open when the apical plug is received within the aperture; and
a lid to be received on the bracket and defining an aperture corresponding with the bracket aperture, within which the apical plug may be received;
wherein the lid is rotatable relative to the bracket so as to align or unalign the second regions of the respective apertures, so as to open or close a path through the lid and bracket.

The cell culture insert may define apical and basal domains of the culture well, with the apical plug sealing the apical domain when received in the cell culture insert. The path through the lid and bracket defined by the second regions of the respective apertures extends past the cell culture insert to allow access to the basal domain of the culture well.

Further features of this aspect of the invention are as defined for the first aspect.

Other features of the invention will be apparent from the accompanying drawings, and the following detailed description.

### BRIEF SUMMARY OF THE DRAWINGS

FIG. 1 is an illustrative sketch of a cell culture insert within a culture well;

FIG. 2 shows a bracket for use in an embodiment of the present system;

FIG. 3 shows a lid for use with the bracket of FIG. 2;

FIG. 4 shows a cell culture system including wells, cell culture inserts, apical plugs, and the bracket and lid of FIGS. 2 and 3.

### DETAILED DESCRIPTION OF THE INVENTION

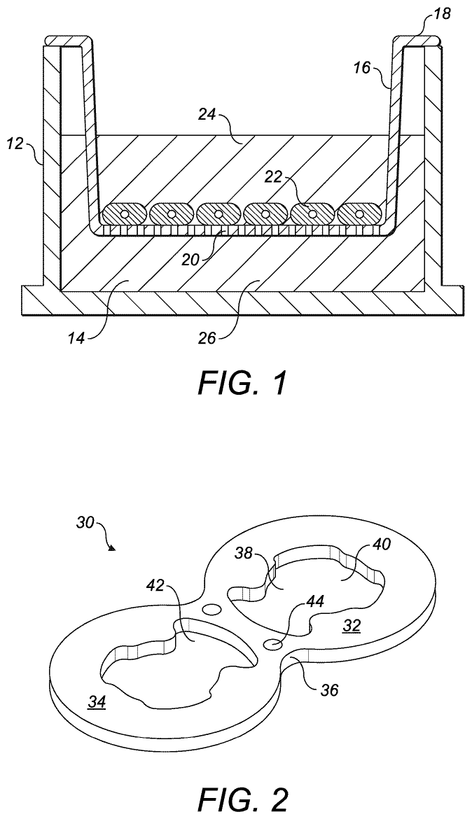

A schematic illustration of a typical cell culture insert cell culture system is shown in FIG. 1. An individual cell culture well 12 (which may be one well within a multiwell culture plate) is filled with a cell culture medium 14, and a cell culture insert 16 located within the well 12. The cell culture insert 16 is designed to be suspended above the lower surface of the well 12 via flanges 18 which engage with the upper surface of the well. Typically, the flanges 18 are discontinuous, thereby allowing materials (such as cell culture medium) to be added to or removed from the culture well outside the cell culture insert 16.

The cell culture insert 16 includes a lower surface 20 formed of a semi-permeable or microporous membrane. A monolayer of cells 22 may be grown on the surface 20 within the cell culture insert. The cell culture insert 16 divides the internal volume of the cell culture well 12 into two domains—an apical domain 24, within the cell culture insert 16, and a basal domain 26, outside the cell culture insert. This allows different conditions to be applied to the apical and basal surfaces of the cell monolayer 22, and the effect of these to be studied. Representative use cases include simulating conditions within and outside the gut, or co-culturing different cell types in different domains of the culture well.

In order to allow for the apical domain 24 to be effectively sealed and separated from the basal domain 26, and for culture medium to be flowed over the apical surface, apical plugs have been developed which fit into the cell culture insert 16 to a shorter depth than the lower surface 20, and engage in a sealing fit with the sides of the cell culture insert via, for example, elastomeric O-rings. The apical plugs include inlet and outlet openings on an upper surface to allow culture medium to access the apical domain 24. In one such system, described in U.S. Pat. No. 10,323,221, the upper surface of the apical plug is sized so as to extend beyond the edges of the cell culture insert 16 and culture well 12, to thereby effectively seal the well as a whole, and hence prevent contamination of both the apical and basal domains.

However, this arrangement means that accessing the basal domain is not possible without removing the apical plug and/or the cell culture insert 16, risking contamination of the basal domain and potentially disrupting a cell monolayer 22 if the cell culture insert is manipulated too aggressively.

The present invention addresses this issue, by providing an alternate means to seal or unseal access to the basal domain, while also permitting the use of an apical plug to provide sealed access to the apical domain.

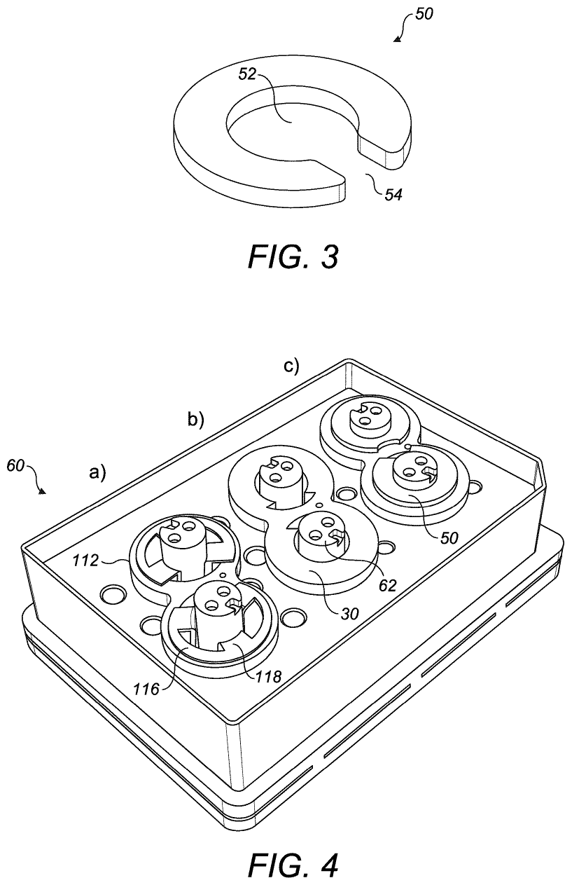

A first element of a cell culture component is shown in FIG. 2, with a further element shown in FIG. 3. A multiwell culture plate incorporating the assembled cell culture component, together with other elements of a cell culture system, is shown in FIG. 4.

FIG. 2 shows a bracket 30 being of a generally figure-eight form, and arranged to sit above a pair of cell culture wells (see FIG. 4). The figure-eight form provides two lobes 32, 34, which are symmetrical about a central portion 36 of the bracket 30, with each lobe 32, 34 sitting above a respective culture well in use.

Each lobe 32, 34 defines an aperture 38 which itself is bilobate, thereby being divided into two contiguous regions. The first such region 40 is centrally-located within its lobe 32, and is sized and shaped to accommodate an apical plug (not shown in FIG. 2). The second region 42 is offset from the centre of the lobe, and extends closer to the rim of the lobe than does the first region.

The central portion 36 of the bracket includes a pair of holes 44 which in use can receive screws to secure the bracket 30 to a cell culture well plate.

FIG. 3 shows a further element of the cell culture component, a lid 50. This is generally annular in shape, having a central aperture 52 which extends into a notch 54 extending through the lid 50, resulting in the lid itself being discontinuous.

The cell culture component is shown in use on FIG. 4. This shows a cell culture plate 60 which includes multiple cell culture wells 112. In the illustration, these are grouped into pairs, for convenience, designated by letters a), b), and c). The first pair, a), shows each well receiving a cell culture insert 116, and within the cell culture insert is received an apical plug 62. The cell culture insert 116 is supported on the rim of the well 112 by means of a number of flanges 118, which both distance the wall of the cell culture insert 116 from that of the well 112, and provide further spaces whereby access to the basal compartment of the well may be maintained. The apical plug 62 seals the apical compartment of the cell culture insert, and includes fluid channels which permit circulation of culture medium through the apical compartment.

In a conventional apical plug system, the plug would include a larger covering which would restrict access to the basal compartment as well. However, as shown in well pair b), here the bracket 30 is placed over the wells. The bracket 30 itself has a wider diameter than that of the cell culture insert 116, while the first portion 40 of the aperture 38 is sized so as to permit the apical plug 62 to be received within it. As is apparent from the Figure, the second portion 42 of the bracket aperture 38 is not at this time sealed, and so access to the basal compartment of the well is maintained. As is also apparent from the Figure, when in use, if the apical plug 62 is to be removed then the bracket 30 may be restrained manually and the plug 62 lifted upwards from the well. The bracket will allow the plug to pass, and the bracket will prevent the cell culture insert 116 from being dislodged, thereby reducing disturbance to the cell culture within.

Finally, as shown in well pair c), in order to seal the access to the basal compartment, the lid 50 can be placed over the upper part of the apical plug 62, and rest on top of the bracket 30. Given the annular nature of the lid 50, this may be freely rotated about the apical plug 62, such that the notch 54 can either align with the second portion 42 of the bracket aperture, providing access to the basal compartment, or can not align with this portion, so effectively closing access to the basal compartment and acting to prevent contamination.

It can be seen that this provides a simple yet effective way to make use of apical plugs and cell culture inserts in a cell culture system, with the ability to access both the apical and basal compartments while preventing contamination without the need for additional covers, and also enabling easier removal or insertion of the apical plug.

1. A cell culture component for use with a cell culture system having a cell culture well, a cell culture insert received in the cell culture well, and an apical plug received in the cell culture insert, the cell culture component comprising:
a bracket defining an aperture within which an apical plug may be received, the bracket having an outer diameter larger than that of the cell culture insert, and the aperture having a diameter smaller than that of the cell culture insert; wherein the aperture is sized and shaped so as to define a first region which will be closed by an apical plug when the plug is received within the aperture, and a second region which will remain open when the apical plug is received within the aperture; and
a lid to be received on the bracket and defining an aperture corresponding with the bracket aperture, within which the apical plug may be received;
wherein the lid is rotatable relative to the bracket so as to align or unalign the second regions of the respective apertures, so as to open or close a path through the lid and bracket.

2. The cell culture component of claim 1, wherein the bracket aperture is asymmetric, such that the second region is offset from the first region.

3. The cell culture component of any preceding claim, wherein the bracket aperture is bilobate.

4. The cell culture component of claim 3 wherein the bracket aperture includes a first, larger region for receiving the apical plug, and a second, offset, smaller region for forming the path.

5. The cell culture component of claim 4 wherein the first and second regions of the bracket aperture are contiguous.

6. The cell culture component of any preceding claim wherein the lid aperture which corresponds with the bracket aperture is not identical to the bracket aperture.

7. The cell culture component of any preceding claim wherein the corresponding regions of the lid aperture and the bracket aperture are of different sizes and/or shapes.

8. The cell culture component of any preceding claim wherein the lid is generally annular.

9. The cell culture component of claim 8 wherein the first lid aperture region is a central annular opening, and the second aperture region is in the form of a notch from the rim of the annulus.

10. The cell culture component of any preceding claim wherein the first regions of the lid aperture and the bracket aperture are concentric.

11. The cell culture component of any preceding claim wherein the bracket is able to be fixed to the culture well.

12. The cell culture component of any preceding claim wherein the bracket comprises a plurality of apertures as defined; preferably two such apertures.

13. The cell culture component of any preceding claim wherein the bracket has a bilobate or FIG. 8 shape.

14. The cell culture component of any preceding claim further comprising one or more of a cell culture well, a cell culture insert, and an apical plug.

15. A cell culture system comprising a cell culture well, a cell culture insert received in the cell culture well, and an apical plug received in the cell culture insert, and further comprising a cell culture component as defined in any preceding claim.

16. A cell culture system comprising a cell culture well, a cell culture insert received in the cell culture well, and an apical plug received in the cell culture insert, the cell system further comprising:
a bracket to be located on the cell culture well and defining an aperture within which the apical plug may be received, the bracket having an outer diameter larger than that of the cell culture insert, and the aperture having a diameter smaller than that of the cell culture insert; wherein the aperture is sized and shaped so as to define a first region which will be closed by the apical plug when the plug is received within the aperture, and a second region which will remain open when the apical plug is received within the aperture; and
a lid to be received on the bracket and defining an aperture corresponding with the bracket aperture, within which the apical plug may be received;
wherein the lid is rotatable relative to the bracket so as to align or unalign the second regions of the respective apertures, so as to open or close a path through the lid and bracket.
